(19) United States
(12) Patent Application Publication
(10) Pub. No.: US 2021/0068989 A1
(43) Pub. Date: Mar. 11, 2021

(54) ARTIFICIAL FOOT

(71) Applicant: MECURIS GMBH, Munich (DE)

(72) Inventors: Jannis BREUNINGER, Grossbettlingen (DE); Franziska GLAS, Munich (DE); Bence ROCHLITZ, Munich (DE); Carolin TAUBMANN, Neuhaus (DE); Clemens RIETH, Boeblingen (DE)

(21) Appl. No.: 17/054,400

(22) PCT Filed: May 8, 2019
(86) PCT No.: PCT/EP2019/061839

§ 371 Date: Nov. 10, 2020

(30) Foreign Application Priority Data
May 14, 2018 (DE) 20 2018 102 690.9

(51) Int. Cl. A61F 2/66; A61F 2/50

(52) U.S. Cl. CPC A61F 2/66; A61F 2/5046; A61F 2002/6657; A61F 2002/6642; A61F 2002/665

(57) ABSTRACT

The invention relates to a prosthetic foot comprising a base body having a front sole region, a heel body having a rear sole region, and a slot configured to receive at least one spring element, in particular at least one leaf spring, the spring element configured to be pushed or inserted into the slot. In a disposed state, the spring element engages in the base body and the heel body such that the heel body is spring mounted relative to the base body.

### CROSS-REFERENCE TO RELATED APPLICATIONS

This application is a U.S. National Phase application submitted under 35 U.S.C. § 371 of Patent Cooperation Treaty application serial no. PCT/EP2019/061839, filed May 8, 2019, and entitled ARTIFICIAL FOOT, which application claims priority to German patent application serial no. 20 2018 102 690.9, filed May 14, 2018, and entitled ARTIFICIAL FOOT.

Patent Cooperation Treaty application serial no. PCT/EP2019/061839, published as WO 2019/219483 A1, and German patent application serial no. 20 2018 102 690.9, are incorporated herein by reference.

### TECHNICAL FIELD

The invention relates to a prosthetic foot for use in a leg or foot prosthesis.

### BACKGROUND

A prosthetic foot is known from DE 20 2015 007 994. Prosthetic feet are exposed to extremely high loads. For this reason, existing prosthetic feet are manufactured from plastics, fiber-reinforced plastics and/or metal alloys using an injection molding process or a lamination process. Prosthetic feet, which are produced using additive manufacturing processes, also known as 3D printing, are known from DE 10 2014 006 727 B3.

There are a number of aspects that are important for fulfilling the functional requirements of a prosthetic foot. The damping properties, which are most evident upon heel impact, are essential to comfort when walking. The geometry of the sole is important for a harmonious rolling behavior during walking. Also desirable, and usually achieved by means of passive spring elements, is an energy return when the forefoot is lifted off the ground. This ensures energy-efficient walking for the prosthesis wearer.

An individual, patient-specific design of the overall prosthesis is generally important in order to adapt the functional elements to the needs of the patient. Conventional prosthetic feet, however, allow only limited individual modifications. For the most part, it is not the prosthetic foot itself that is modified. Rather the certified prosthetist/orthotist selects a foot that best matches the desired properties from the many different prosthetic feet. Prosthetic feet are usually also available in different sizes. Some can still be adapted, for example, by exchanging or adjusting the damping elements.

The additively manufactured prosthetic feet currently still in development offer the possibility of individually adapting each individual prosthetic foot using tool-free production. Thus, not only the foot size can be adjusted here, but also, to a particularly high degree, the damping properties and the rolling behavior. However, the materials available for these processes do not currently provide the spring properties and thus the energy return that can be achieved, for example, with conventionally manufactured, fiber-reinforced plastics.

### SUMMARY

Based on this state of the art, it is the object of the present invention to provide an improved prosthetic foot, in particular an improved foot prosthesis. A further object is to design the prosthetic foot in such a way that a higher degree of individualization is made possible in a simple and cost-effective manner. The production costs should be as low as possible and the comfort of the user should be as high as possible.

This object is achieved by the subject matter according to claim 1.

The object is in particular achieved by a prosthetic foot comprising a base body having a front sole region and a heel body having a rear sole region. The prosthetic foot can comprise a slot for receiving at least one (additional) spring element which can be pushed or inserted into said slot. The additional spring element can be bar springs or leaf springs. According to the invention, in a disposed state, the additional spring element engages in the base body and the heel body such that the heel body is spring mounted relative to the base body.

A core aspect of the invention is thus to create a prosthetic foot, in which essential structural elements are individually produced in a simple manner, in particular in an additive process, whereby the behavior of the prosthetic foot when walking or running is adjusted by means of individually selectable spring elements. The selection of the spring elements can be based on weight specifications and/or requirement profiles.

The front and/or rear sole region does not necessarily have to be made of rubber. According to the invention, it merely refers to a region that correlates to the biological sole of the foot. The prosthetic foot according to the invention can be designed in such a way that it is worn openly, i.e. without the use of a cosmetic. However, it is also possible to apply the thinking of the present invention to a prosthetic foot that is used in combination with a cosmetic.

According to the invention, the concept of spring mounting used above can be defined in such a way that, in order to move the heel body, in particular in horizontal direction, a spring force that is exerted by said spring element is overcome.

In one embodiment, the additional spring element is connected to the base body. It is possible for the spring element to “float” in an opening or recess of the base body and/or the heel body. There is preferably an attachment, wherein the attachment can be established using an adhesive connection and/or a screw connection and/or a latching connection and/or a connection via a press fit. Reversible attachments are preferred. A fixed connection can be provided in at least one region and/or in two regions, e.g. in the front and in the center or in the rear and in the center. In one embodiment, it can be provided that the spring element is “floatingly” disposed in two regions, e.g. in the front and in the center or in the rear and in the center or in the front and in the rear. In one embodiment, the spring element can be connected to the base body (only) in a front region and/or in a rear region via an attachment. In one embodiment, it is also possible for the spring element to be connected to the base body in a front and a rear region. This creates an arrangement in which the spring element is fixed and not “floating”.

In one embodiment, the spring element rests centrally on the base body, which produces an axis of rotation or a range of rotation at this contact point about which the spring element can rotate. As a result of this central contact, the pressure on the heel (springing away) when the prosthetic foot comes down bends the front and/or central region of the prosthetic foot in such a way that the central region of the prosthetic foot comes into contact with the ground relatively early. This type of deformation of the plastic makes the prosthetic foot particularly comfortable to use.

In one embodiment, the connection between the spring element and the base body is located in the central region as well.

According to the invention, centrally or central region can be defined in such a way that this is a region near the center of the spring element. For example, this region can comprise a region that is at most 20%, in particular at most 10%, of the total length from the center of the spring element.

In one embodiment, the additional spring element can be mounted in the front region of the base body and/or in the rear region of the heel body, in particular in an opening, so as to be movable in the longitudinal direction of the spring element.

As already mentioned, the spring element can be an elongated spring element, for example a leaf spring or a spring bar. Theoretically, it is also possible to use a plurality of leaf springs or a plurality of spring bars. The spring can be made of a fiber-reinforced plastic. However, other materials such as resilient metals, in particular spring steel, or plastics without fibers are conceivable too.

It is advantageous if at least sections of the prosthetic foot can move relative to the spring element in the longitudinal direction of the spring element. This displaceability allows the prosthetic foot to deform even more when walking or running, thus making placing the foot on the ground dynamic and comfortable.

This displaceability is preferably achieved by mounting end sections of the spring element in openings. These openings are preferably configured such that they adjoin the spring element in vertical direction with or without play. In horizontal direction, with respect to the longitudinal axis of the spring element, there is space, so that the spring element can move inside the opening. In the heel body, said opening can be the aforementioned slot.

The base body can comprise a forefoot or forefoot body with the front sole region and a mounting body, in particular for receiving an adapter. Such adapters are known, and have in some cases already been standardized, to allow the prosthetic foot to be attached to the user's body quickly and effectively by suitable means.

The forefoot body can be flexibly connected to the mounting body, in particular via a web. The forefoot body can thus move relative to the adapter and absorb and release forces. Therefore the flexible connection in the form of a web is supported by the structure of the prosthetic foot. The web thus performs the function of a spring element.

In one embodiment, the heel body is connected to the base body via a (further) web. This web preferably extends substantially (max. ±30°) in horizontal direction, so that the heel element can compress in vertical direction. The web can extend parallel to the additional spring element at least in sections. In one embodiment, the web surrounds the spring element at least along one section. The cross-section of the web can be an (inverted) U-profile or C-profile, for example. It is possible for this web to also perform the function of a spring element. Ultimately, the flexibility of the heel body in relation to the base body can be adjusted by appropriately dimensioning the web and the spring element. In one embodiment, the materials that are used to produce the web and that form the additional spring element have different spring properties. A targeted selection of the dimensions allows highly individualized rolling curves to be set.

The front sole region can have a convex shape at least in sections, preferably over at least 50% or 30% of the contact surface. The contact surface can be defined such that it is the surface that comes into contact with the ground during a normal walking or running movement on a level surface.

The base body can comprise a stop element that extends downward. This stop element can abut the spring element directly or indirectly. The stop element is preferably configured such that the region with which the stop element indirectly or directly abuts the spring element is in the front region, in particular in the front third of the spring element. The stop element transmits forces acting on the spring element into the adapter. When the foot is placed on the ground, the spring can rotate around the region in which the stop element abuts said spring.

The stop element can extend substantially from the adapter element toward the front and down. In one embodiment, a longitudinal axis of the stop element is inclined approx. 30° relative to an adapter plane.

The additional spring element can be disposed substantially parallel to the adapter plane and/or in a plane which rises toward the front. According to the invention, the ascending arrangement can include an angle of 1-5°, preferably 4.4°, in particular 1-3° particularly preferably 1.8°. The angle of the spring element relative to the ground plane is preferably 1-10°. The spring element is thus relatively close to the ground in the rear region, whereas, in the front region, it is much further away. In one embodiment, the prosthetic foot is configured such that the spring element is disposed relatively close to the ground; i.e. no more than 3 cm away from the ground.

In one embodiment, the angle between the additional spring element and the ground plane can depend on the height of the heel body. For example, a heel body height of 12 mm can result in an angle of 4.4°. It is thus possible to adapt the prosthetic foot to a shoe in which the prosthetic foot can be worn such that, when the prosthetic foot is inside the shoe, the angle between a ground plane and the additional spring element is at least substantially approximately 0° (±2°.

The base body can comprise a rear stop, which is spaced apart from the heel body by a gap. The gap thus allows the heel body to vibrate or flex relative to the base body, in particular relative to the stop. However, at maximum load, the heel body abuts the stop, so that damage to the prosthetic foot can be avoided. The stop is therefore preferably relatively solid.

In one embodiment, the stop is configured such that, at maximum load, the heel body abuts the stop over a larger area, at least 1 cm2, preferably at least 2 cm2.

The prosthetic foot can be produced in an additive process. The prosthetic foot is preferably produced using 3D printing. It is possible to use a thermoplastic.

The base body and/or heel body and/or forefoot body and/or mounting body and/or at least one of the webs and/or the stop element and/or other elements can be produced integrally, i.e. as one element.

The aforementioned object is also achieved by a method for producing an prosthetic foot having the features already described.

Further advantageous embodiments emerge from the subclaims.

### BRIEF DESCRIPTION OF THE DRAWINGS

The invention is described in the following using several design examples, which are explained in more detail with the aid of illustrations. The figures show:

FIG. 1 a first prosthetic foot, wherein the base body and the heel body are connected via a web;

FIG. 2 a second prosthetic foot, in which the base body and heel body are connected only via a leaf spring;

FIG. 3 a third prosthetic foot;

FIG. 4 a longitudinal section through the first prosthetic foot of FIG. 1;

FIG. 5 a first perspective view of the prosthetic foot of FIG. 1 (from above);

FIG. 6 a second perspective view of the prosthetic foot of FIG. 1 (from below);

FIG. 7 an exemplary side view of a leaf spring;

FIG. 8 a fourth prosthetic foot.

### DETAILED DESCRIPTION

In the following description, the same reference numbers are used for parts that are the same or have the same effect.

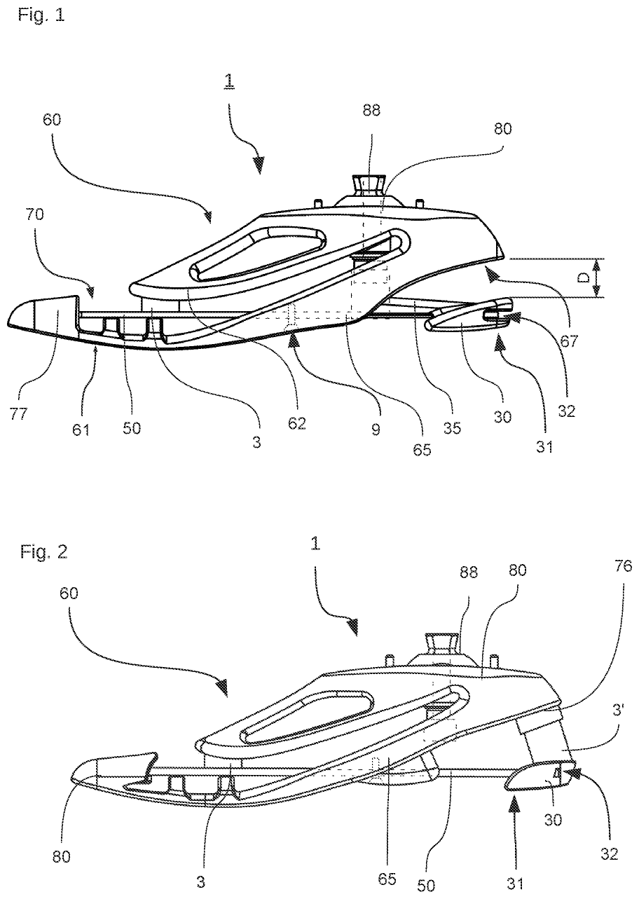

FIG. 1 shows a side view of a prosthetic foot 1 according to the invention. Said foot is substantially composed of two elements, namely a heel body 30 and a base body 60. In the design example shown in FIG. 1, the heel body 30 and the base body 60 are connected to one another via a heel web 35. The heel web extends substantially horizontally. The base body 60, heel web 35 and heel body 30 form an integral unit, which is produced in an additive process, for example by 3D printing.

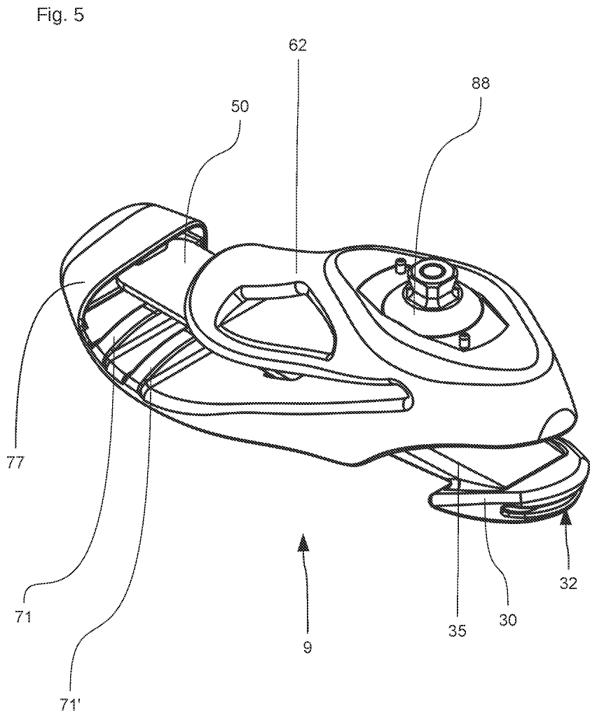

The heel body 30 comprises a slot 32 having a corresponding slot opening, into which a leaf spring 50 is inserted. The leaf spring 50 extends in the longitudinal direction of the prosthetic foot 1, substantially horizontally to a ground plane BE (FIG. 4). The length of the leaf spring 50 corresponds substantially to the total length of the prosthetic foot. It is only 15% shorter than the total length of the prosthetic foot 1. The leaf spring 50 reinforces the heel web 35 and provides a spring mounting of the heel body 30.

The rear part of the base body 60 with a rear stop 67 is noticeably spaced apart from the heel body. There is gap there, having a gap height D of at least 1 cm. In the shown design example, the gap height D is 2 cm. The gap allows free movement of the heel body 30 in vertical direction. In the event of overloading, the heel body 30 strikes the rear stop 67, and thus prevents the heel web 35 and/or the leaf spring 50 from being subjected to excessive loads that could possibly cause it to break.

The base body 60 can be functionally subdivided into further elements. These are a forefoot body 70, a mounting body 80, a web 65 and a stop element 62. All of these elements are integrally connected to one another. The web 65 provides an elastic connection between the mounting body 80 and the forefoot body 70. On its upper side, the mounting body 80 holds an adapter 88. The web 65 extends substantially from top right to bottom left and merges smoothly into the forefoot body 70. The web 65 has an average thickness of approx. 2 cm. According to the invention, the web 65 can have an average thickness of 1 to 5 cm, in particular 1 to 3 cm.

A front sole region 61 is located on the underside of the forefoot body 70. A corresponding rear sole region 31 is located on the underside of the heel body 30. In the design example shown in FIG. 1, the rear sole region 31 and the front sole region 61 are provided with a rubber coating to ensure good ground contact.

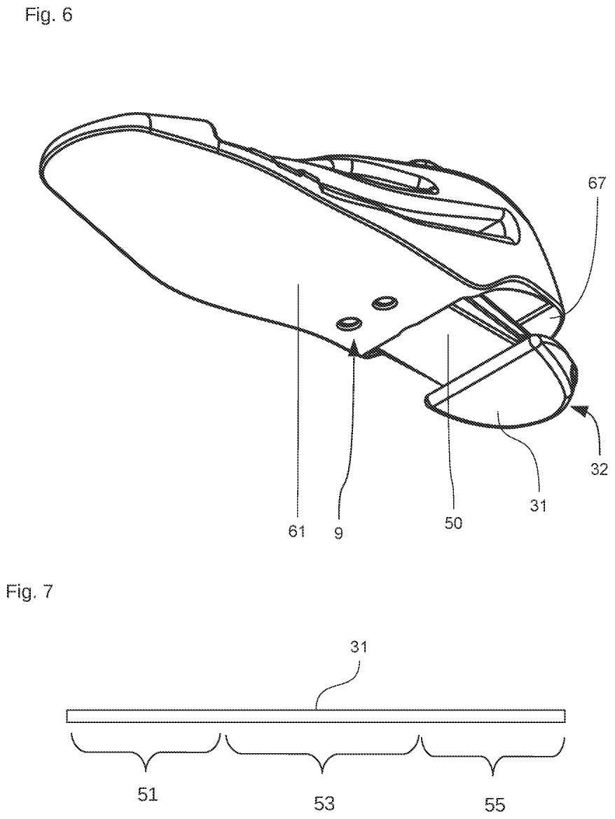

The forefoot body 70 further comprises a cap 77 having a receptacle (see FIG. 4) into which the leaf spring 50 projects. In the center of the prosthetic foot 1, at the level of the web 65, the leaf spring 50 is screwed to the base body 60 with two screws 9 (see also FIG. 6). The screws 9 secure the leaf spring 50 against displacement in longitudinal direction. At its ends, the leaf spring 50 is not connected to the prosthetic foot and projects loosely into the openings provided there, so that, for example, the cap 77 can move in longitudinal direction relative to the leaf spring 50 when walking. The leaf spring 50 is supported with respect to the forefoot body 70 via two transverse webs 71, 71′ (see FIG. 3). The first, front transverse web 71 and the second, rear transverse web 71′ are integral components of the forefoot body 70. They can in turn be configured as spring elements or have a resilient effect.

The stop element 62 forms the upper region of the base body 60 and extends substantially parallel to the web 65. The web 65 and the stop element 62 are spaced apart from one another. This results in a gap of approx. 1 cm, which extends upward toward the rear beyond the load line and/or construction line of the prosthetic foot 1. The gap extends substantially (approx. +/−15%) in a 45 degree angle to the longitudinal axis of the leaf spring 50.

A damper 3 which rests directly on the upper side of the leaf spring 50 is disposed in the front lower region of the stop element 62. The second transverse web 71′ starts at this location on the underside of the leaf spring 50. The second transverse web 71′ and the stop point or stop region of the stop element 62 are thus substantially located at the same position in longitudinal direction.

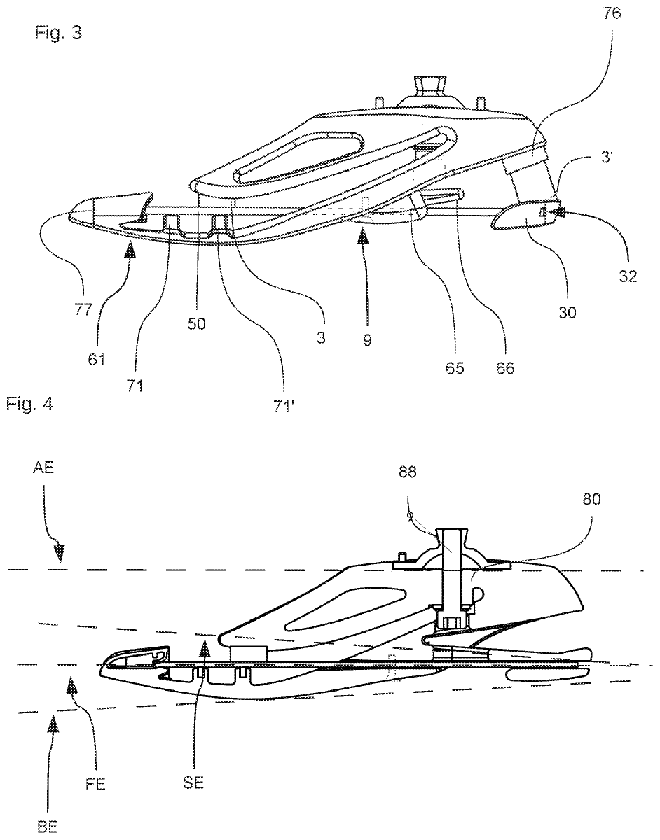

FIG. 2 shows a further design example of the prosthetic foot 1 according to the invention. The heel web 35 of FIG. 1 has been omitted here. The heel body 30 is instead configured as a separate element. The heel body 30 and the base body 60 are therefore not integrally connected to one another. The leaf spring 50 is substantially disposed in the same way as already explained for the prosthetic foot 1 of FIG. 1. According to the invention, additional mounting means can be provided in the heel body 30 to prevent a displacement of the heel body 30 in the longitudinal direction of the spring element 50. The heel body 30 of FIG. 2 also has a slot 32, in which the spring element 50 is inserted. The heel body 30 is also spring mounted such that movement in vertical direction is possible. A damper 3′, which is connected to the stop 76, attaches to the upper side of the heel body 30. In one embodiment, the heel body 30 does not have any additional mounting means. The longitudinal displaceability of the heel body 30 relative to the base body 60 is limited by the damper 3 and by the stop 76.

FIG. 3 shows a prosthetic foot 1, which corresponds substantially to the prosthetic foot of FIG. 2.

One difference is that the web 65 comprises a tab 66. The tab 66 is disposed above the leaf spring 50 and extends substantially in the direction of the heel body 30 horizontally to the ground plane. In the shown design example, the tab 66 extends over approx. 40% of the distance between the web 65 and the heel body 30. If the leaf spring 50 is deformed in vertical direction, the tab 66 acts as a stop element, so that the movement of the leaf spring 50 is limited at least over the length of the tab 66. Under load, the leaf spring 50 lies flat against the tab 66.

FIG. 4 shows a cross-section through the prosthetic foot 1 of FIG. 1. Relevant planes that are necessary for specifying the position of the leaf spring 50 and the heel web 35 can be defined with the aid of this cross-section. The adapter 88, for example, with its mounting points on the base body 60, defines an adapter plane AE that extends substantially horizontally. A ground plane BE extends substantially parallel (approx. −20°) to the adapter plane AE. The spring plane FE, in which the leaf spring 50 is located, extends substantially parallel to the adapter plane AE. In the shown design example, the spring plane rises toward the front by approx. 5°. The heel web 35 extends along a heel web plane FE. This heel web plane FE is significantly more inclined relative to the adapter plane AE and the ground plane BE than the spring plane FE. The resulting angle is between 10 and 15°.

FIGS. 5 and 6 show further perspective views of the prosthetic foot 1 of FIG. 1.

FIG. 7 shows a leaf spring 50 according to the invention, wherein said spring is subdivided into three sections; namely a front region 51, a central region 53 and a rear region 55. The front region 51 and the rear region 55 substantially occupy a quarter of the total length of the leaf spring 50. The central region extends over approx. two quarters of the total length of the leaf spring 50. The already discussed attachment by means of the screws 9 is carried out in the central region 53. The transverse webs 71, 71′ abut the leaf spring 50 in the transition area from the front region 51 to the central region 53. In this region, the stop element 62 also abuts the leaf spring 50 via the damper 3.

The rear region 55 is largely occupied by the slot 52 of the heel body 30.

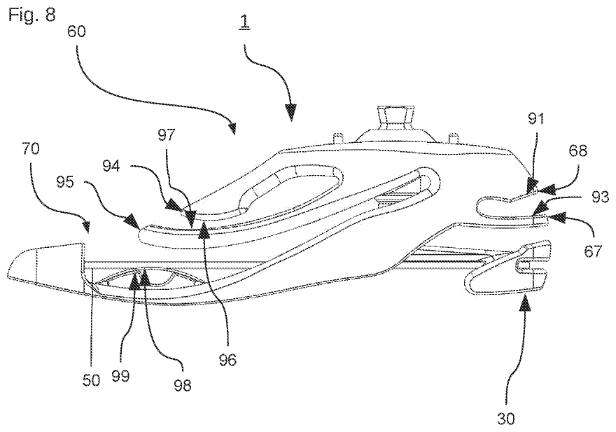

FIG. 8 shows a further embodiment of a prosthetic foot 1, which corresponds substantially to the prosthetic feet 1 of FIGS. 1 to 7.

In contrast to the prosthetic feet of FIGS. 1 to 3, the prosthetic foot 1 of FIG. 8 comprises a damped stop 67 in the rear region of the prosthetic foot 1. The stop 67 comprises a substantially flat lower stop region 93, which, under load, is pressed against an upper stop region 91 of a rear end stop 68. Corresponding to the upper stop region 91, the lower stop region 93 is configured such that, under load, the lower stop region 93 comes into planar contact with the upper stop region 91. This ensures a uniform introduction of force into the prosthetic foot 1.

In the unloaded state, the lower stop region 93 extends at least substantially parallel to a ground plane. When the heel body 30 is loaded, it is pressed against the rear stop 67, so that it acts as a spring and is pressed against the rear end stop 68.

The prosthetic foot 1 further comprises a web 98, via which the leaf spring 50 is supported relative to the forefoot body 70. The web 98 is bent over a web bracket 99, so that the leaf spring 50 always remains in contact with the web 98 even when the forefoot body 70 is deformed.

In the design example of FIG. 8, the stop element 62 of the base body 60 is subdivided in the front region (toward the forefoot), so that a further damping effect is provided. In the design example of FIG. 8, the stop element 62 comprises an upper stop region 94 and a lower damping region 95. The lower damping region 95 is disposed above the leaf spring 50, so that the leaf spring 50 is pressed against the lower damping region 95 when the forefoot body 70 is subjected to a load during a walking motion. The lower damping region 95 is flexible, so that the lower damping region 95 is pressed against the upper stop region 94 under load. This provides better overall damping properties, which results in greater wearing comfort for the user.

At this point, it should be noted that all of the parts described above, alone and in any combination, in particular the details shown in the drawings, are claimed as being essential to the invention. For example, numerous variations with respect to the inclination angle of the heel web 35 and/or the leaf spring 50 are conceivable. Similarly, the leaf spring 50 can be replaced by one or a plurality of leaf springs 50 and/or spring webs.

### REFERENCE SIGNS

1 Prosthetic foot
3, 3′ Damper
9 Screw
30 Heel body
31 Rear sole region
32 Slot
35 Heel web
50 Leaf spring
51 Front region of the leaf spring
53 Central region of the leaf spring
55 Rear region of the leaf spring
60 Base body
61 Front sole region
62 Stop element
65 Web
66 Tab
67 Rear stop
68 Rear end stop
70 Forefoot body
71, 71′ Transverse web
77 Cap
80 Mounting body
88 Adapter
91 Upper stop region
93 Lower stop region
94 Upper stop region
95 Lower damper section
96 Lower damper surface
97 Upper damper surface
98 Web
99 Web bracket
AE Adapter plane
FE Spring plane
SE Heel web plane
BE Ground plane
D Gap height

1. A prosthetic foot comprising:
a base body having a front sole region;
a heel body having a rear sole region; and
a slot configured to receive at least one spring element, the spring element configured to be pushed or inserted into the slot,
wherein, in a disposed state, the spring element engages in the base body and the heel body such that the heel body is spring mounted relative to the base body.

2. The prosthetic foot according to claim 1, wherein:
the spring element is connected centrally to the base body using one of an adhesive connection, a screw connection, a latching connection, or a press fit connection.

3. The prosthetic foot according to claim 1, wherein:
the spring element is mounted in at least one of an opening in a front region of the base body or an opening in a rear region of the heel body so as to be movable in a longitudinal direction of the spring element.

4. The prosthetic foot according to claim 1, wherein:
the base body comprises a forefoot body and a mounting body, the front sole region located on an underside of the forefoot body, and
the mounting body is configured to receive an adapter.

5. The prosthetic foot according to claim 4, wherein:
the forefoot body is flexibly connected to the mounting body via a web.

6. The prosthetic foot according to claim 1, wherein:
the heel body is connected to the base body via a web, wherein the web is configured such that the web surrounds the spring element at least in sections.

7. The prosthetic foot according to claim 1, wherein:
the front sole region has a convex shape at least in sections, over at least 40% of a contact surface of the front sole region.

8. The prosthetic foot according to claim 1, wherein:
the base body comprises a stop element which extends downward at an angle of 30 to 60 degrees to a longitudinal axis of the spring element, and which rests on the spring element.

9. The prosthetic foot according to claim 1, wherein:
the spring element, in the disposed state, is disposed in a plane that is substantially parallel to an adapter plane, or is disposed in a plane that rises toward a front of the prosthetic foot.

10. The prosthetic foot according to claim 1, wherein:
the base body comprises a rear stop which is spaced apart from the heel body by a gap.

11. The prosthetic foot according to claim 1, wherein the prosthetic foot is produced in an additive process.

12. The prosthetic foot according to claim 1, wherein:
at least two of the base body, the heel body, a forefoot body, a mounting body, a web that flexibly connects the forefoot body to the mounting body, or a stop element that rests on the spring element formed in one piece.

13. The prosthetic foot according to claim 1, wherein:
the spring element comprises fiber-reinforced plastic.

14. The prosthetic foot of claim 1, wherein:
the spring element comprises GFRP or CFRP.

15. The prosthetic foot of claim 1, wherein:
the spring element is a leaf spring.

16. The prosthetic foot of claim 2, wherein:
the spring element connected centrally to the base body is connected to the base body at a point within a region of the spring element that comprises 10% of a total length from a center of the spring element.

17. The prosthetic foot of claim 9, wherein:
the adapter plane is defined by mounting points on the base body that are configured to mount an adapter.

18. The prosthetic foot of claim 10, wherein:
the gap is at least 1 cm.

19. The prosthetic foot of claim 11, wherein:
the additive process is a 3D printing process.
